(19) United States
(12) Patent Application Publication
(10) Pub. No.: US 2026/0242309 A1
(43) Pub. Date: Aug. 20, 2026

(54) HIGHLY EFFICIENT AND STABLE LYCOPENE CIS-ISOMERIZATION METHOD

(71) Applicant: CHINA PHARMACEUTICAL UNIVERSITY, Jiangsu (CN)

(72) Inventors: Chongjiang CAO, Jiangsu (CN); Linyu NIAN, Jiangsu (CN)

(73) Assignee: CHINA PHARMACEUTICAL UNIVERSITY, Jiangsu (CN)

(21) Appl. No.: 19/646,586

(22) Filed: Apr. 14, 2026

Related U.S. Application Data
(63) Continuation of application No. PCT/CN2025/140654, filed on Dec. 8, 2025.

(30) Foreign Application Priority Data
Dec. 9, 2024 (CN) 202411797462.0
Nov. 28, 2025 (CN) 202511775792.4

(51) Int. Cl. C07C 5/22

(52) U.S. Cl. CPC C07C 5/2286; C07C 2531/38

(57) ABSTRACT

The present disclosure discloses a highly efficient and stable method for cis-isomerization of lycopene, where a bimetallic metal-organic framework material is used as a catalyst to catalyze lycopene to undergo cis-isomerization. In the method, confined water molecules may be further added to the reaction system as a catalytic medium to achieve highly efficient catalysis for isomerization of lycopene. The catalyst attacks and activates the conjugated double bonds of all-trans lycopene in an electron-rich environment, thereby enabling rotation to form the corresponding configuration. The confined water molecules serve as an electron transfer medium, accelerating the ability of trans-lycopene to lose electrons to form a lycopene radical intermediate during isomerization, thereby further improving the yield of cis-isomerization of lycopene.

### CROSS-REFERENCES TO RELATED APPLICATIONS

This application is a continuation of PCT application No. PCT/CN2025/140654, filed on Dec. 8, 2025, which claims priority right of Chinese patent application No. 202411797462.0 filed on Dec. 9, 2024, and Chinese patent application No. 202511775792.4 filed on Nov. 28, 2025. The entirety of each of the above-mentioned patent applications is hereby incorporated by reference herein and made a part of this specification.

### BACKGROUND

### Technical Field

The present disclosure relates to a highly efficient and stable method for cis-isomerization of lycopene.

### Description of Related Art

90% or more of lycopene in natural plants exists in the all-trans configuration, and its bioavailability, antioxidant activity, and other physiological activities are lower than those of the cis configuration. In particular, the 5-cis-isomer exhibits higher biological activity and stability, and therefore has broad prospects for development and application in the fields of food, nutritional products, and pharmaceuticals. These characteristics make lycopene products containing a high proportion of cis-isomers possess higher added value in the market. Therefore, the development of highly selective, highly efficient, and highly stable cis-isomerization technologies is of key significance for the preparation of high-value-added nutritional fortifiers.

Common methods for cis-isomerization of lycopene include thermal isomerization, photo-induced isomerization, catalyst-mediated isomerization, microwave isomerization, electrochemical isomerization, and the like. Thermal/photo-induced isomerization promotes the conversion of lycopene from all-trans-isomers to cis-isomers through direct heating, and is currently the most commonly used isomerization method in research. In particular, during thermal treatment, an increase in temperature and an extension of processing time increase the proportion of cis-isomers; however, this also results in significant degradation of lycopene. Therefore, such isomerization strategies are generally limited by inherent problems of low conversion efficiency (generally <50%) and strong reversibility.

In the prior art, heterogeneous catalysts such as I—TiO₂ and Cu-MCM-41 are applied to lycopene isomerization reactions, but the reactions generally need to be performed in pure organic solvents, such as dichloromethane or acetone. Such systems not only exhibit low reaction rates and limited product yields (for example, the yield is about 50% after 6 h), but also cause degradation of lycopene by organic solvents, resulting in product loss with a degradation rate of up to 30%. To improve reaction efficiency, recent studies have attempted to introduce a small amount of free water into the organic medium so as to reduce the reaction energy barrier by means of water molecules, thereby improving the isomerization efficiency. For example, when 2.5% water is added to an acetone system catalyzed by FeCl₃ or AlCl₃, the yield of total cis-lycopene can be increased to 5 times the original yield. Furthermore, the Fe₂(SO₄)₃₋ free water composite catalytic system increases the cis-isomerization efficiency to 65% by forming [Fe(H₂O)₆]³⁺ hydrated ions, reconstructing the hydrogen bond network, and enhancing the electron coupling effect between the metal ions and the lycopene π system. However, in this system, the coordination stability of the hydrated ions is insufficient, and lycopene undergoes self-aggregation due to hydrophobic interactions, forming a lamellar structure that hinders effective mass transfer, resulting in a final product retention rate of only 52%.

### SUMMARY

Objective: The objective of the present disclosure is to provide a highly selective, highly efficient, and stable method for cis-isomerization of lycopene.

Technical solution: The present disclosure discloses a highly efficient and stable method for cis-isomerization of lycopene, where a bimetallic metal-organic framework material is used as a catalyst to catalyze lycopene to undergo cis-isomerization.

The bimetallic metal-organic framework material is formed by doping a zirconium-based organic framework material with a transition metal material, the crystal structure of the bimetallic metal-organic framework material is octahedral and formed by coordination between an organic ligand tetrafluoroterephthalic acid and metal ions, and the metal ions are zirconium ions, copper ions, zinc ions, or iron ions; among the metal ions, copper ions, zinc ions, or iron ions are doped metal ions, and the molar amount of the doped metal ions is 20-50% of the total molar amount of the metal ions.

The zirconium-based organic framework material includes UIO-66 or MOF-801, and the bimetallic metal-organic framework material is Zn_{1/5}—UIO-66, Zn_{1/4}—UIO-66, Zn_{1/2}—UIO-66, Cu_{1/5}—UIO-66, Cu_{1/4}—UIO-66, Cu_{1/2}—UIO-66, Fe_{1/5}—UIO-66, Fe_{1/4}—UIO-66, Fe_{1/2}—UIO-66, or Cu-MOF-801.

A method for preparing the bimetallic metal-organic framework material includes the following steps:
(1) dissolving zirconyl nitrate together with one of copper nitrate trihydrate, zinc nitrate hexahydrate, and iron nitrate nonahydrate in a solution of methanol and formic acid, and stirring for dissolution to obtain a mixed solution, where the molar ratio of the zirconyl nitrate to one of the copper nitrate trihydrate, the zinc nitrate hexahydrate, and the iron nitrate nonahydrate is 4:1 to 1:1, and the volume ratio of the methanol to the formic acid is 5:1 to 2:1;
(2) adding tetrafluoroterephthalic acid into the mixed solution, and stirring at a temperature of 30° C.-50° C. for 10 h-14 h; and
(3) subjecting a solid material obtained to centrifugal washing and vacuum drying to obtain the bimetallic metal-organic framework material, where methanol is used as a washing agent, and the centrifugation is performed at a rotating speed of 3000 rpm/min for 10 min at a temperature of 10° C.-20° C.; the vacuum drying is performed at a temperature of 45° C.-60° C. for 8 h-10 h under a drying pressure of −0.09 MPa to −0.1 MPa.

The method of the present disclosure specifically includes using the bimetallic metal-organic framework material and a lycopene solution, stirring, performing heating under reflux in a water bath, and allowing to react in the dark; and filtering a lycopene mixed solution after the reaction to obtain a product, where the ratio of an amount of the bimetallic metal-organic framework material used to lycopene used is 4:1 to 2:1, the heating is performed at a temperature of 25° C.-50° C. for 0.5 h-2 h, magnetic stirring is employed, heating under reflux in a water bath is performed, and the reaction is performed in the dark.

According to the method of the present disclosure, trans-lycopene is dissolved in an organic solvent, and a catalytic medium may be further added to form a catalytic system to perform cis-isomerization preparation of lycopene, where the catalytic medium is confined water, and the volume percentage of the catalytic medium in the catalytic system is 1%-2%.

The catalytic medium is confined water, that is, water molecules confined within the cavities and channels of the porous structure of Cu-MOF-801; the water molecules are restricted within molecular-scale or nanoscale spaces to form a confinement effect, and the volume percentage of the confined water in the catalytic system is 1%-2%.

The method for preparing the bimetallic catalyst includes the following steps:
(1) dissolving trans-lycopene in acetone, adding the bimetallic metal-organic framework material into the reaction system, and adding the catalytic medium into the reaction system to form a catalytic system, where due to the confinement effect of the synthesized transition-metal-based catalyst, water molecules can be precisely regulated through coordination interactions, and the catalytic medium water molecules exist in the form of confined water molecules;
(2) subjecting the above catalytic system to water-bath heating and stirring; and
(3) centrifuging a solution after completion of catalysis to recover a catalyst precipitate, thereby obtaining a solution of cis-lycopene in acetone and completing isomerization of lycopene.
In step (1), the bimetallic metal-organic framework material is a Cu-doped MOF-801 nanomaterial, and the concentration of the bimetallic metal-organic framework material is 0.5 mg/mL.
In step (2), the heating is performed at a temperature of 30° C.-40° C., and the stirring is performed for 1 h.

The method for preparing the bimetallic catalyst includes: dissolving fumaric acid, zirconium oxychloride octahydrate, and copper nitrate trihydrate in a mixed solution of DMF and formic acid, and thoroughly stirring and uniformly mixing to obtain a mixed solution; allowing the mixed solution to react at a high temperature, and after completion of the reaction, cooling and washing at room temperature to obtain a precipitate; and subjecting the precipitate to vacuum drying to obtain the bimetallic catalyst.

The molar mass of fumaric acid is 4 mmol-6 mmol, the molar mass of zirconium oxychloride octahydrate is 3.54 mmol, the molar mass of copper nitrate trihydrate is 1 mmol-5 mmol, and the volume ratio of DMF to formic acid is (18 mL-20 mL):(5 mL-8 mL); the reaction is performed at a high temperature of 120° C.-130° C. for 5 h-6 h; the drying is performed at a temperature of 50° C.-60° C. for 7 h-8 h, and the vacuum drying is performed under a pressure of −0.1 MPa.

The method for preparing the bimetallic metal-organic framework material specifically includes:
(1) dissolving 5 mmol of fumaric acid, 3.75 mmol of zirconium oxychloride octahydrate, and 1.25 mmol of copper nitrate trihydrate in a mixed solution of 20 mL of DMF and 7 mL of formic acid, and further thoroughly stirring and uniformly mixing the mixed solution;
(2) transferring the above mixed solution to a high-pressure reactor, placing the reactor in an oven (130° C., 6 h), and after completion of the reaction, cooling at room temperature; then performing centrifugal washing four times with DMF and methanol; and
(3) drying the washed precipitate in a vacuum drying oven (50° C., 8 h), and performing vacuum drying by using a vacuum pump until the pressure reaches-0.1 MPa, thereby obtaining a Cu-doped MOF-801 transition metal-based nanomaterial.

Inventive principle: The highly efficient and stable method for isomerization of lycopene of the present disclosure and the method for promoting cis-isomerization of lycopene of the present disclosure utilize a transition metal (Cu, Fe)-doped bimetallic metal-organic framework material as a catalyst to achieve the isomerization of all-trans lycopene and improve its biological activity. The catalyst has the ability to accept electron pairs and thus acts as a Lewis acid. All-trans lycopene is rich in conjugated double bonds, which create an electron-rich environment and are susceptible to attack by electrophiles, thereby acting as a Lewis base. Therefore, the conjugated double bonds of all-trans lycopene can be readily attacked and activated by the catalyst, thereby enabling rotation to form the corresponding configuration. The doping of an appropriate amount of Cu and Fe improves the ability of the material to accept electrons, such that the optimal catalytic effects of the bimetallic metal-organic framework materials Cu_{1/4}@UIO-66 and Fe_{1/2}@UIO-66 are achieved. Therefore, Cu_{1/4}@UIO-66 and Fe_{1/2}@UIO-66 exhibit overall superior performance in retention rate, total isomerization rate, and 5-position isomerization rate compared with undoped UIO-66, Zn-doped UIO-66, and UIO-66 doped with other amounts of Cu or Fe, which are greener and more efficient.

Further, transition metal-confined water synergistic catalysis is employed to catalyze isomerization of lycopene. Through a transition-metal-based catalyst, the occurrence state of water molecules is regulated, such that the water molecules are transformed from free water to nano-confined water. The confined water molecules serve as an electron transfer medium in the isomerization pathway of trans-lycopene, accelerating the ability of trans-lycopene to lose electrons to form a lycopene radical intermediate during isomerization, thereby improving the yield of cis-isomerization of lycopene. Studies have shown that the above catalytic process involves a competitive relationship between electronic effects and mass-transfer limitations: the metal-water interface promotes rearrangement of the π-electron cloud, which is beneficial for the isomerization reaction; by regulating the occurrence form of water molecules in the reaction environment, the problem that aggregation behavior induced by free water severely restricts the transmission efficiency of reactants at the catalytic interface is overcome, the reaction pathway is optimized, and the bottleneck in the prior art in which efficiency and selectivity are difficult to achieve simultaneously is broken through, thereby providing a new approach for achieving highly efficient cis-isomerization of lycopene.

In addition, the isomerization efficiency and retention rate of lycopene are further improved by optimizing the catalyst concentration and the catalytic time. Excessively high catalyst concentration and excessively long catalytic time may increase the degradation rate of lycopene, whereas excessively low catalyst concentration and excessively short catalytic time cannot achieve the isomerization of trans-lycopene. Therefore, under the condition that confined water molecules serve as a medium to improve efficiency, multiple conditions are mutually matched, and a synergistic enhancement effect is achieved by optimizing the catalytic medium content, catalyst concentration, and catalytic time, such that the optimal effect of lycopene isomerization is achieved.

Beneficial effects: Compared with the prior art, the present disclosure has the following significant advantages: (1) In the method for isomerization of lycopene of the present disclosure, a bimetallic metal-organic framework material is used as a catalyst and confined water is used as a catalytic medium, which is environmentally friendly; and (2) the method of the present disclosure can increase the efficiency of cis-isomerization of lycopene to up to 82.2%, which is far higher than the thermal isomerization efficiency of 37.6% in the prior art; and under the process conditions, the retention rate of lycopene can reach up to 90.3%, with higher catalytic stability.

### BRIEF DESCRIPTION OF THE DRAWINGS

FIG. 1 is a graph of isomerization ratios of lycopene catalyzed by UIO-66 doped with different types and proportions of metals;

FIG. 2 is a liquid chromatogram of lycopene isomerization catalyzed by UIO-66;

FIG. 3 is a diagram of cell viability assay results of HUVEC cells;

FIG. 4 is a diagram of cell viability assay results of NIH 3T3 cells;

FIG. 5 is a fluorescence image of cell viability;

FIG. 6 is a diagram of hemolysis assay results of materials;

FIG. 7 is a liquid chromatogram of catalysis of trans-lycopene with catalytic medium contents;

FIG. 8 is a graph of the isomerization rate and retention rate of catalysis of trans-lycopene with catalytic medium contents;

FIG. 9 is a liquid chromatogram of catalysis of trans-lycopene at different material concentrations;

FIG. 10 is a graph of the isomerization rate and retention rate of catalysis of trans-lycopene at different material concentrations;

FIG. 11 is a liquid chromatogram of trans-lycopene at different catalytic times;

FIG. 12 is a graph of the isomerization rate and retention rate of catalysis of trans-lycopene at different material concentrations;

FIG. 13 is an SEM image of lycopene with different cis-lycopene contents;

FIG. 14 is an XRD image of lycopene with different cis-lycopene contents; and

FIG. 15 is an in situ variable-temperature X-ray diffraction pattern of binding of catalyst to water molecules.

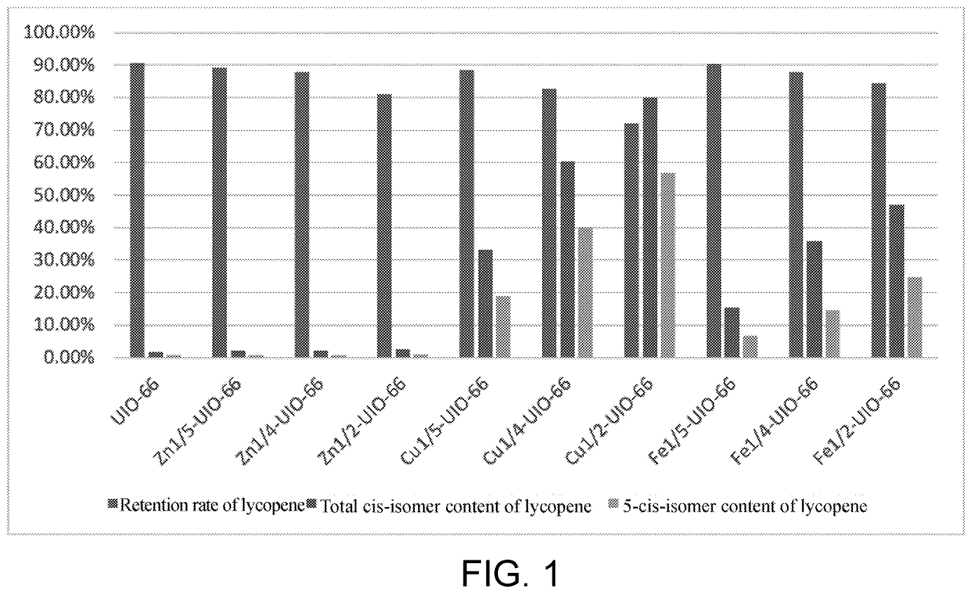

### DESCRIPTION OF THE EMBODIMENTS

The technical solutions of the present disclosure are further described below in conjunction with examples. The experimental materials used in the examples can be obtained through conventional commercial sources.

### Example 1

In the method for promoting cis-isomerization of lycopene of the present disclosure, a Cu_{1/4}—UIO-66 material was used as a catalyst to catalyze lycopene to undergo cis-isomerization.

### Preparation of Cu_{1/4}—UIO-66 Material:

(1) 0.045 mmol of zirconyl nitrate and 0.015 mmol of copper nitrate trihydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the bimetallic metal-organic framework material Cu_{1/4}—UIO-66 of the present disclosure was obtained.

The method for cis-isomerization of lycopene specifically includes the following steps: 1 mg of the bimetallic metal-organic framework material and 10 mL of a lycopene solution (0.1 mmol/L) were added into a round-bottom flask, the mixture was incubated at 37.5° C. for 1 h with magnetic stirring and heated under reflux in a water bath, and the reaction was performed in the dark.

### Example 2

Compared with Example 1, in the method for promoting cis-isomerization of lycopene, the catalyst was replaced with iron ion-doped Fe_{1/2}—UIO-66, and the doping molar amount was 50% of the total molar amount of the metal ligand.

### Preparation of Fe_{1/2}—UIO-66 Material:

(1) 0.03 mmol of zirconyl nitrate and 0.03 mmol of iron nitrate nonahydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached −0.1 MPa, such that the bimetallic metal-organic framework material Fe_{1/2}—UIO-66 of the present disclosure was obtained.

### Example 3

Compared with Example 1, in the method for promoting cis-isomerization of lycopene, the catalyst was replaced with zinc ion-doped Zn_{1/5}—UIO-66, and the doping molar amount was 20% of the total molar amount of metal ions.

### Preparation of Zn_{1/5}—UIO-66 Material:

(1) 0.048 mmol of zirconyl nitrate and 0.012 mmol of zinc nitrate hexahydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the bimetallic metal-organic framework material Zn_{1/5}—UIO-66 of the present disclosure was obtained.

### Example 4

Compared with Example 1, in the method for promoting cis-isomerization of lycopene, the catalyst was replaced with zinc ion-doped Zn_{1/4}—UIO-66, and the doping molar amount was 25% of the total molar amount of metal ions.

### Preparation of Zn_{1/4}—UIO-66 Material:

(1) 0.045 mmol of zirconyl nitrate and 0.015 mmol of zinc nitrate hexahydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the bimetallic metal-organic framework material Zn_{1/4}—UIO-66 of the present disclosure was obtained.

### Example 5

Compared with Example 1, in the method for promoting cis-isomerization of lycopene, the catalyst was replaced with zinc ion-doped Zn_{1/2}—UIO-66, and the doping molar amount was 50% of the total molar amount of metal ions.

### Preparation of Zn_{1/2}—UIO-66 Material:

(1) 0.03 mmol of zirconyl nitrate and 0.03 mmol of zinc nitrate hexahydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the bimetallic metal-organic framework material Zn_{1/2}—UIO-66 of the present disclosure was obtained.

### Example 6

Compared with Example 1, in the method for promoting cis-isomerization of lycopene, the catalyst was replaced with copper ion-doped Cu_{1/5}—UIO-66, and the added molar amount was 20% of the total molar amount of metal ions.

### Preparation of Cu_{1/5}—UIO-66 Material:

(1) 0.048 mmol of zirconyl nitrate and 0.012 mmol of copper nitrate trihydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the bimetallic metal-organic framework material Cu_{1/5}—UIO-66 of the present disclosure was obtained.

### Example 7

Compared with Example 1, in the method for promoting cis-isomerization of lycopene, the catalyst was replaced with copper ion-doped Cu_{1/2}—UIO-66, and the added molar amount was 50% of the total molar amount of metal ions.

### Preparation of Cu_{1/2}—UIO-66 Material:

(1) 0.03 mmol of zirconyl nitrate and 0.03 mmol of copper nitrate trihydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the bimetallic metal-organic framework material Cu_{1/2}—UIO-66 of the present disclosure was obtained.

### Example 8

Compared with Example 1, in the method for promoting cis-isomerization of lycopene, the catalyst was replaced with iron ion-doped Fe_{1/5}—UIO-66, and the doping molar amount was 20% of the total molar amount of metal ions.

### Preparation of Fe_{1/5}—UIO-66 Material:

(1) 0.048 mmol of zirconyl nitrate and 0.012 mmol of iron nitrate nonahydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the bimetallic metal-organic framework material Fe_{1/5}—UIO-66 of the present disclosure was obtained.

### Example 9

Compared with Example 1, in the method for promoting cis-isomerization of lycopene, the catalyst was replaced with iron ion-doped Fe_{1/4}—UIO-66, and the doping molar amount was 25% of the total molar amount of metal ions.

### Preparation of Fe_{1/4}—UIO-66 Material:

(1) 0.045 mmol of zirconyl nitrate and 0.015 mmol of iron nitrate nonahydrate were weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the bimetallic metal-organic framework material Fe_{1/4}—UIO-66 of the present disclosure was obtained.

### Comparative Example 1

Compared with Example 1, in the method for promoting cis-isomerization of lycopene, the catalyst was replaced with a UIO-66 material without metal-ion doping:

### Preparation of UIO-66 Material:

(1) 0.06 mmol of zirconyl nitrate was weighed out and dissolved in 7 mL of methanol and 1.5 mL of acetic acid, and the mixture was stirred at 40° C. until complete dissolution.
(2) To the above solution was added 1.429 mL of a solution of tetrafluoroterephthalic acid in methanol (10 mg/mL). The mixture was stirred at 40° C. for 12 h, and then centrifugally washed with methanol at 3000 rpm/min three times, each time for 1 min.
(3) The washed precipitate was dried in a vacuum drying oven (50° C., 8 h), and vacuum drying was performed by using a vacuum pump until the pressure reached-0.1 MPa, such that the metal-organic framework material UIO-66 of the present disclosure was obtained.

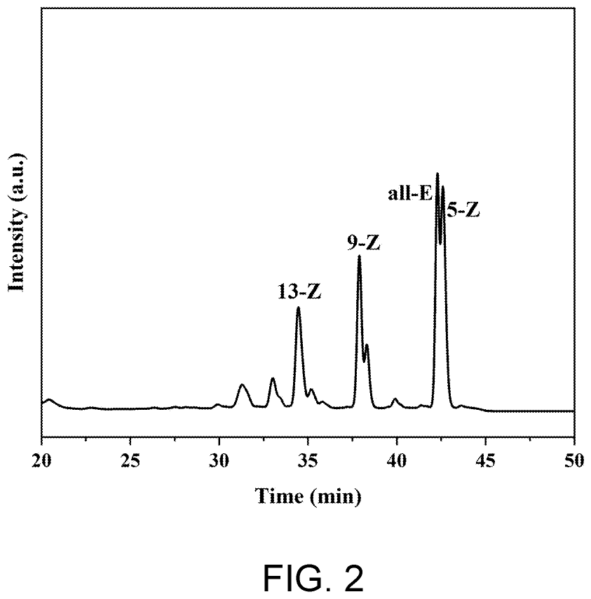

In the method for cis-isomerization of lycopene of Examples 1-9 and the isomerization method of Comparative Example 1, the mixed solution of lycopene after the reaction was filtered through a 0.22 μm polytetrafluoroethylene (PTFE) membrane filter and then subjected to high-performance liquid chromatography analysis. As shown in FIG. 2, the chromatographic peak with a retention time of 34 min corresponded to 13-cis-lycopene, 38 min corresponded to 9-cis-lycopene, 42 min corresponded to all-trans lycopene, and 43 min corresponded to 5-cis-lycopene. The calculation methods for total cis-isomer content (%), individual isomer content (%), and lycopene retention rate (%) are as follows:

$$Total cis - isomer content ( \% ) = \frac{Total peak area of cis - isomers}{Total peak area of lycopene} \times 100$$

$$Individual isomer content ( \% ) = \frac{Total peak area of an individual isomer}{Total peak area of lycopene} \times 100$$

$$Retention rate of lycopene ( \% ) = \frac{Total peak area of lycopene after reaction}{Total peak area of lycopene before reaction} \times 100$$

The isomerization results are shown in Table 1:

TABLE 1
Lycopene cis-isomerization results

| | | Total | 5-cis- |
|---|---|---|---|
| | Retention | isomerization | isomerization |
| | rate | rate | rate |
| UIO-66 | 90.62% | 1.82% | 0.21% |
| Zn1/5-UIO-66 | 89.23% | 2.03% | 0.41% |
| Zn1/4-UIO-66 | 87.88% | 1.98% | 0.38% |
| Zn1/2-UIO-66 | 81.11% | 2.56% | 0.88% |
| Cu1/5-UIO-66 | 88.45% | 33.00% | 18.87% |
| Cu1/4-UIO-66 | 82.63% | 60.30% | 40.20% |
| Cu1/2-UIO-66 | 72.12% | 79.87% | 56.78% |
| Fe1/5-UIO-66 | 90.45% | 15.32% | 6.78% |
| Fe1/4-UIO-66 | 87.98% | 35.89% | 14.61% |
| Fe1/2-UIO-66 | 84.47% | 46.95% | 24.75% |

As shown in Table 1, the retention rate of Cu_{1/4}—UIO-66 was 82.63%, the total isomerization rate was 60.30%, and the 5-cis-isomerization rate was 40.20%; and the retention rate of Fe_{1/2}—UIO-66 was 84.47%, the total isomerization rate was 46.95%, and the 5-cis-isomerization rate was 24.75%. Based on a comprehensive analysis of the retention rate, the total isomerization rate, and the 5-cis-isomerization rate, it can be seen that Cu_{1/4}—UIO-66 and Fe_{1/2}—UIO-66 exhibit optimal performance in the cis-isomerization of lycopene. This is because doping of an appropriate amount of Fe and Cu improves the ability of UIO-66 to accept electrons, resulting in a higher cis-isomerization rate of lycopene.

Biosafety Study of Cu_{1/4}—UIO-66 and Fe_{1/2}—UIO-66 in the Above Examples:

Preferably, human umbilical vein endothelial cells (HUVECs) and mouse embryonic fibroblasts (NIH 3T3) were used to perform cytotoxicity assays for Cu_{1/4}—UIO-66 and Fe_{1/2}—UIO-66. The culture medium for both cells was DMEM, and the cells were cultured at 37° C. in humidified air containing 5% CO₂.

The cytotoxicity of HUVECs and NIH 3T3 cells was assessed using CCK-8. The cells were seeded in a 96-well plate, and a DMEM medium containing triple antibiotics was added. The cells were then cultured for 24 h. Subsequently, the original culture medium was discarded, and a fresh culture medium containing Cu_{1/4}—UIO-66 and Fe_{1/2}—UIO-66 (0.2 mg/mL, 0.1 mg/mL) was added. After further incubation and growth for 24 h, the cells were washed three times with PBS to remove adhered substances. A fresh culture medium containing 10% CCK-8 was then added. The cells were incubated at 37° C. for 0.5 h, and the absorbance at 450 nm was measured using a microplate reader to determine the relative cell viability.

An AO/PI dual kit was used to determine live and dead cells. Specifically, the cell culture and material incubation procedures were the same as those in the CCK-8 assay, except that the plate was a 14-well plate and the seeding density was 1×10⁵. In addition, considering the actual concentration used in practical applications of the material, the material incubation concentration was modified (0.5 mg/mL). After the incubation was completed, a staining reagent was added, and cell fluorescence was recorded under a fluorescence inverted microscope (Nikon 80i).

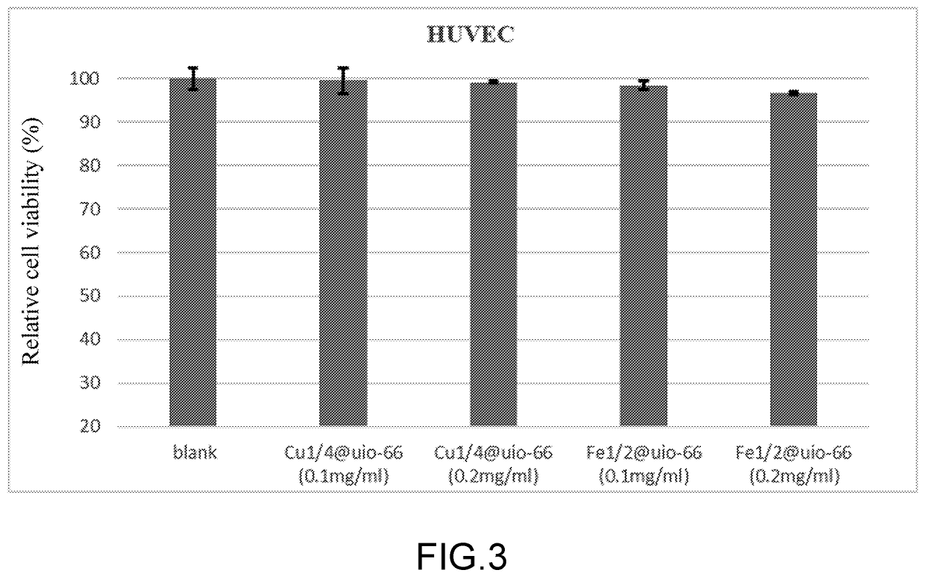

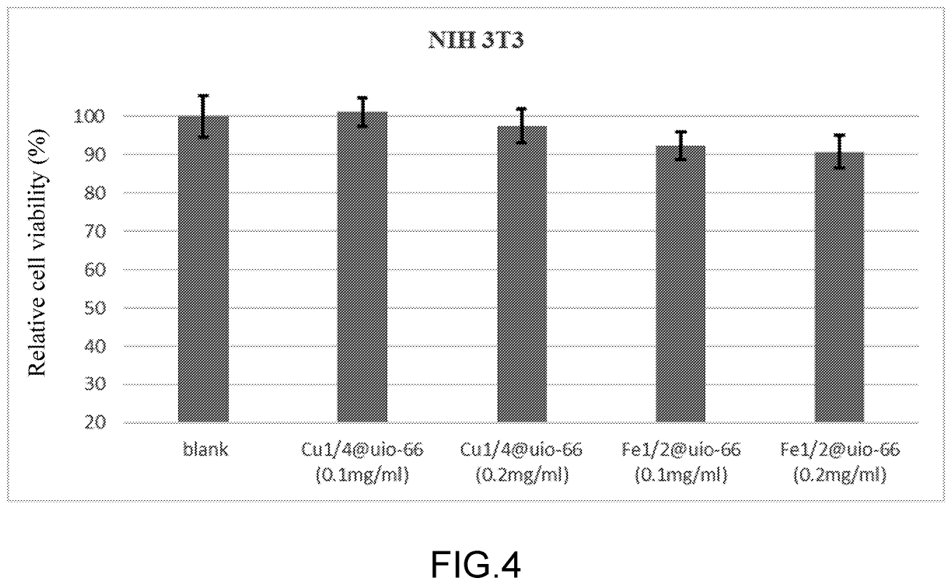

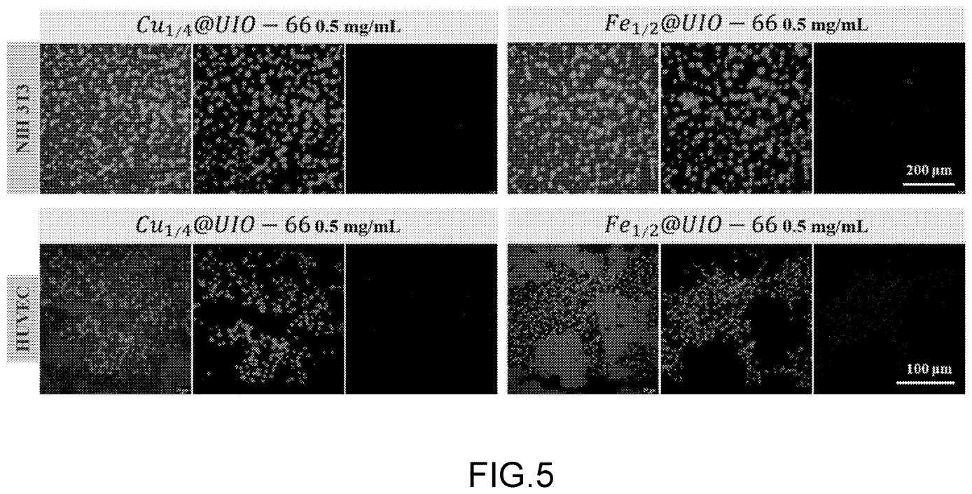

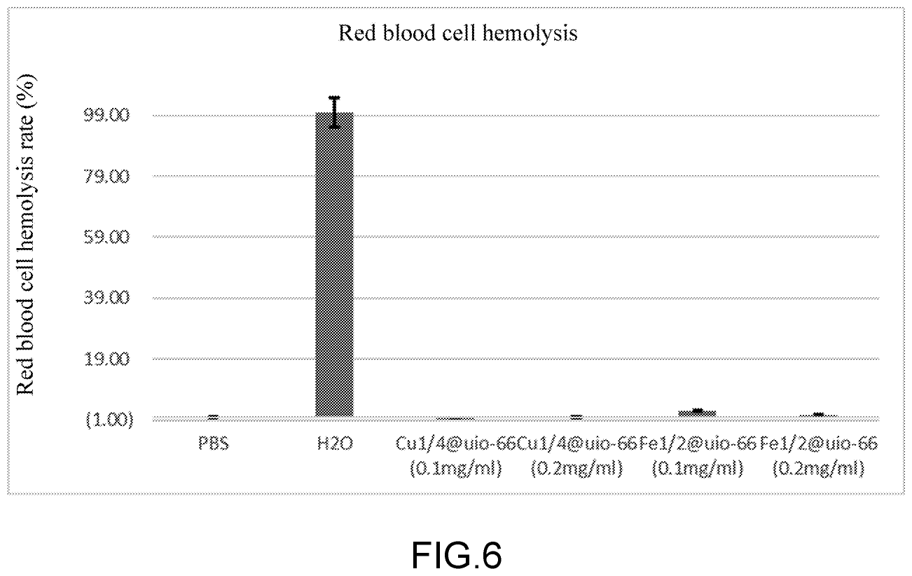

As shown in FIGS. 3 and 4, the results of the CCK-8 cell viability assay indicated that when the material concentration reached 0.2 mg/mL, the survival rates of HUVECs and NIH 3T3 cells still remained 80% or more. Moreover, the fluorescence experiment on live and dead cells also verified that viable cells of the material remained green fluorescence after incubation, while dead cells had almost no red fluorescence (FIG. 5). The experimental results were consistent with the CC-K8 assay results. Meanwhile, the results of the mouse red blood cell hemolysis assay (FIG. 6) showed that the hemolysis rates corresponding to Cu_{1/4}—UIO-66 and Fe_{1/2}—UIO-66 at 0.2 mg/ml were far lower than 5%. In summary, Cu_{1/4}—UIO-66 and Fe_{1/2}—UIO-66 exhibit high biosafety.

### Example 10

In the highly efficient and stable method for isomerization of lycopene of the present disclosure, the bimetallic metal-organic framework material is a Cu-doped MOF-801 transition metal-based nanomaterial, and 1% confined water is added to the catalytic system. The method specifically includes the following steps:
(1) 10 mg of trans-lycopene was weighed out and dissolved in 20 mL of an acetone solution, 10 mg of the bimetallic metal-organic framework material was weighed out and added to the reaction system, the catalyst concentration was 0.5 mg/mL, and 200 μL of confined water was further added to the reaction system to form a catalytic system;
(2) the above catalytic system was transferred to a water bath at 37° C. and magnetically stirred at 600 rpm/min for 1 h; and
(3) the solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

The method for preparing the bimetallic metal-organic framework material specifically includes the following steps: 5 mmol of fumaric acid, 3.75 mmol of zirconium oxychloride octahydrate, and 1.25 mmol of copper nitrate trihydrate were dissolved in a mixed solution of 20 mL of DMF and 7 mL of formic acid, and the mixed solution was further thoroughly stirred and uniformly mixed; the above mixed solution was transferred to a high-pressure reactor, the reactor was placed in an oven (130° C., 6 h), and after completion of the reaction, the mixture was cooled at room temperature. Then, centrifugal washing was performed four times with DMF and methanol; the washed precipitate was dried in a vacuum drying oven (50° C., 8 h), and vacuum drying was performed by using a vacuum pump until the pressure reached-0.1 MPa, thereby obtaining the Cu-doped MOF-801 transition metal-based nanomaterial.

### Example 11

Compared with Example 10, in the highly efficient and stable method for isomerization of lycopene of the present disclosure, 2% of confined water is added to the catalytic system, and the method specifically includes the following steps:
(1) 10 mg of trans-lycopene was weighed out and dissolved in 20 mL of an acetone solution, 10 mg of the bimetallic metal-organic framework material was weighed out and added to the reaction system, the catalyst concentration was 0.5 mg/mL, and 400 μL of confined water was further added to the reaction system to form a catalytic system;
(2) the above catalytic system was transferred to a water bath at 37° C. and magnetically stirred at 600 rpm/min for 1 h; and
(3) the solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

### Comparative Example 2

Compared with Example 10, the content of water molecules was 0%.
(1) 10 mg of trans-lycopene was weighed out and dissolved in 20 mL of an acetone solution, and 10 mg of the bimetallic metal-organic framework material was weighed out and added to the reaction system.
(2) The above reaction system was transferred to a water bath at 37° C. and magnetically stirred at 600 rpm/min for 1 h.
(3) The solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

### Comparative Example 3

Compared with Example 10, the content of water molecules was 5%.
(1) 10 mg of trans-lycopene was weighed out and dissolved in 20 mL of an acetone solution, 10 mg of the bimetallic metal-organic framework material was weighed out and added to the reaction system, and 1000 μL of water was further added to the reaction system to form a catalytic system.
(2) The above catalytic system was transferred to a water bath at 37° C. and magnetically stirred at 600 rpm/min for 1 h.
(3) The solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

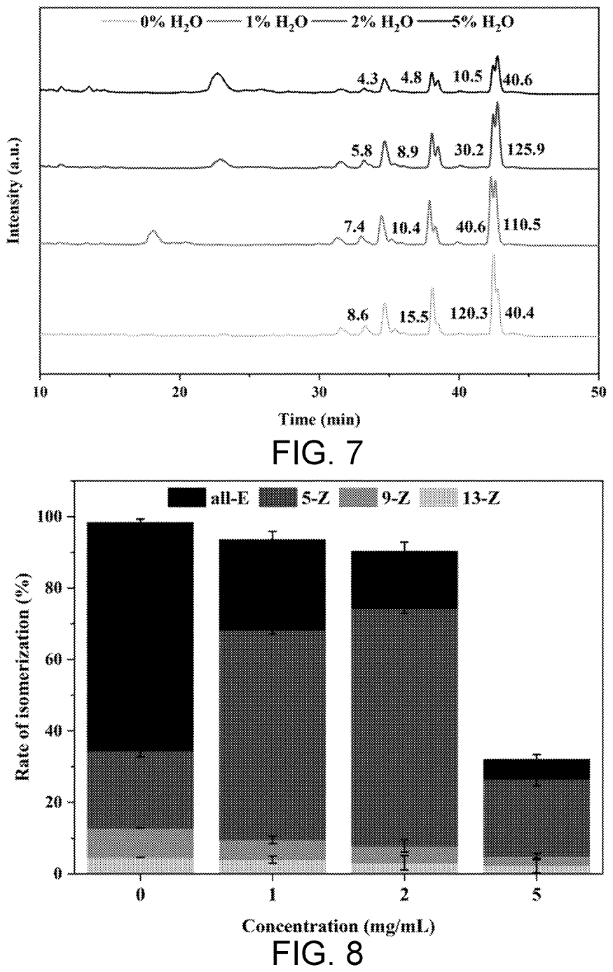

As shown in FIGS. 7 and 8, when 1% and 2% confined water molecules were added to the catalytic system, the proportion of cis-lycopene was increased from 34.9% (no water molecules in the system) to 72.9% and 82.2%, respectively, and the retention rates were 93.6% and 90.3%, respectively. However, when the amount of water molecules was increased to 5%, the retention rate of lycopene remained only 32%. Therefore, adding 1% or 2% confined water molecules in the catalytic system was more conducive to the isomerization of trans-lycopene, and particularly when 2% confined water was added, the isomerization effect was optimal.

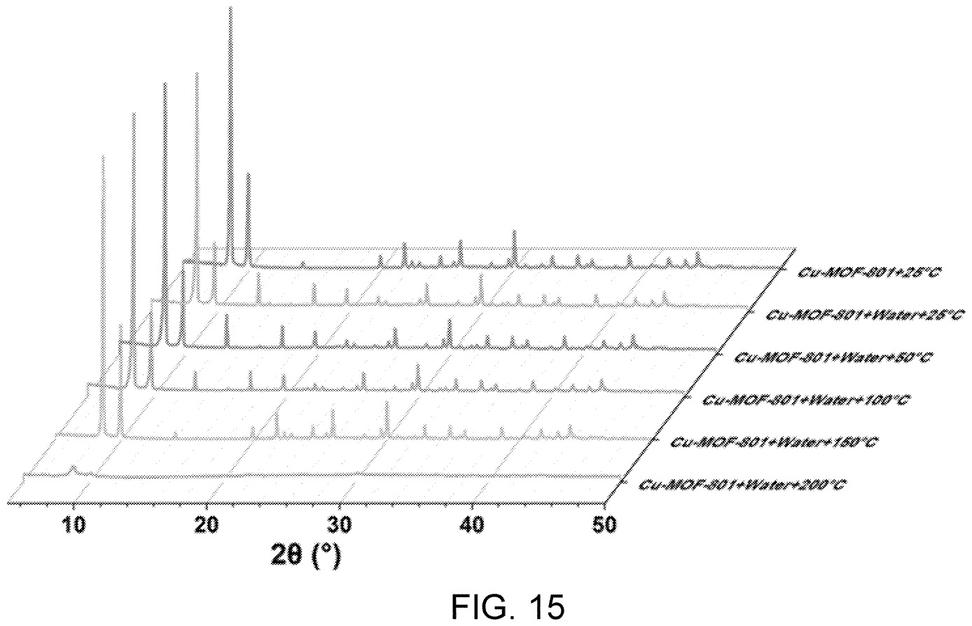

The form of the “confined water” was confirmed by in situ variable-temperature X-ray diffraction analysis. The results in FIG. 15 showed that after Cu-MOF-801 absorbed water, its XRD pattern changed significantly (for example, new peaks appeared at 2θ=13.1° and 17.3°, and characteristic peaks at 22.1° and 24.5° disappeared), indicating that water molecules coordinated with Cu²⁺, resulting in reconstruction of the local crystal structure. This demonstrates that, in Cu-MOF-801, water molecules exist in a “confined state” in which they specifically bind to metal sites.

### Comparative Example 4

Compared with Example 10, the concentration of the bimetallic metal-organic framework material was changed to 0 mg/mL.
(1) 10 mg of trans-lycopene was weighed out and dissolved in 20 mL of an acetone solution, and 200 μL of confined water was added to the reaction system to form a catalytic system.
(2) The above catalytic system was transferred to a water bath at 37° C. and magnetically stirred at 600 rpm/min for 1 h.
(3) The solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

### Comparative Example 5

Compared with Example 10, the concentration of the bimetallic metal-organic framework material was changed to 0.1 mg/mL.
(1) 10 mg of trans-lycopene was weighed out and dissolved in 20 mL of an acetone solution, 2 mg of the bimetallic metal-organic framework material was weighed out and added to the reaction system, and 200 μL of confined water was further added to the reaction system to form a catalytic system.
(2) The above catalytic system was transferred to a water bath at 37° C. and magnetically stirred at 600 rpm/min for 1 h.
(3) The solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

### Comparative Example 6

Compared with Example 10, the concentration of the bimetallic metal-organic framework material was changed to 1 mg/mL.
(1) 10 mg of trans-lycopene was weighed out and dissolved in 20 mL of an acetone solution, 20 mg of the bimetallic metal-organic framework material was weighed out and added to the reaction system, and 200 μL of confined water was further added to the reaction system to form a catalytic system.
(2) The above catalytic system was transferred to a water bath at 37° C. and magnetically stirred at 600 rpm/min for 1 h.
(3) The solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

### Comparative Example 7

Compared with Example 10, the catalytic time was changed to 2 h.
(1) 10 mg of trans-lycopene was weighed out and dissolved in 20 mL of an acetone solution, 10 mg of the bimetallic metal-organic framework material was weighed out and added to the reaction system, and 200 μL of confined water was further added to the reaction system to form a catalytic system.
(2) The above catalytic system was transferred to a water bath at 37° C. and magnetically stirred at 600 rpm/min for 2 h.
(3) The solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

### Comparative Example 8

Compared with Example 10, the catalytic time was changed to 0.5 h.
(1) 10 mg of trans-lycopene was weighed out and dissolved in 20 mL of an acetone solution, 10 mg of the bimetallic metal-organic framework material was weighed out and added to the reaction system, and 200 μL of confined water was further added to the reaction system to form a catalytic system.
(2) The above catalytic system was transferred to a water bath at 37° C. and magnetically stirred at 600 rpm/min for 0.5 h.
(3) The solution after completion of catalysis was centrifuged at 8000 rpm/min, and the catalyst precipitate was recovered. Therefore, a solution of cis-lycopene in acetone was obtained, which was stored at 4° C. in the dark for subsequent liquid chromatography detection.

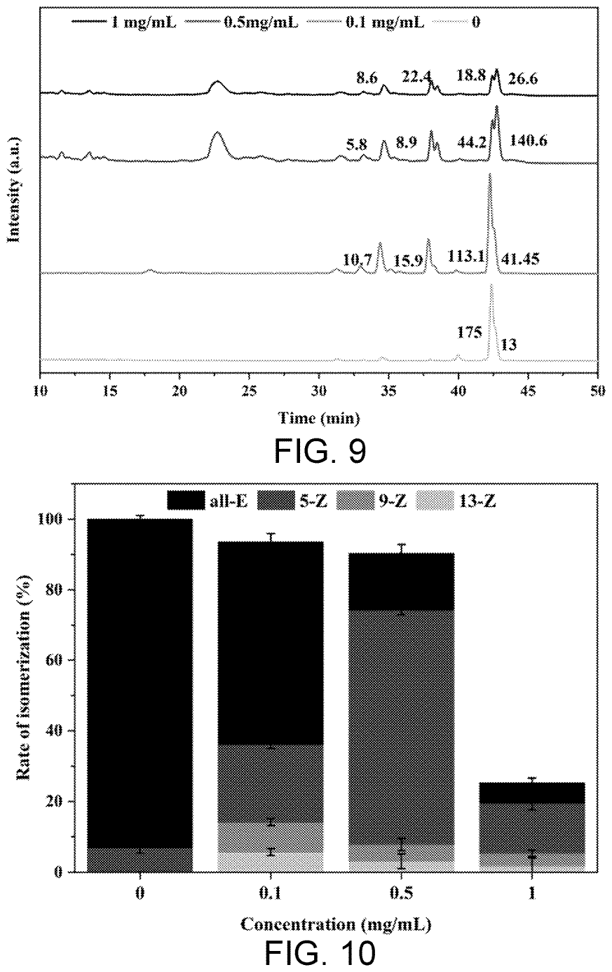

As shown in FIGS. 9 and 10, when the catalyst concentration was 0.1 mg/mL, the isomerization rate was 37.5% and the retention rate was 96.2%, and when the catalyst concentration was 0.5 mg/mL, the isomerization rate was 82.2% and the retention rate was 90.3%. However, when the catalyst concentration was increased to 1 mg/mL, the cis-isomerization rate of lycopene was only 77.2%, and the lycopene retention rate was decreased to 25.2%. Therefore, when the catalyst concentration was 0.5 mg/mL, the isomerization effect was optimal.

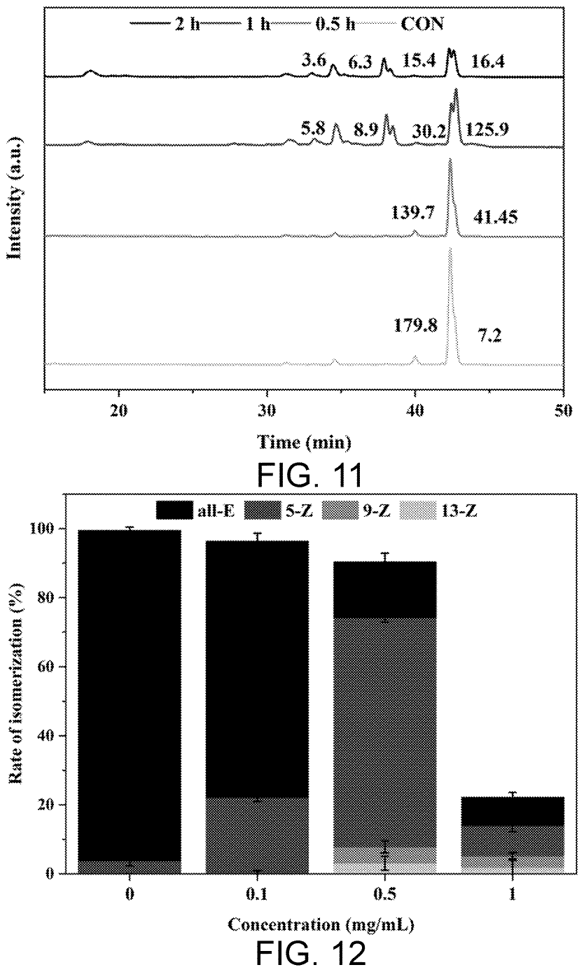

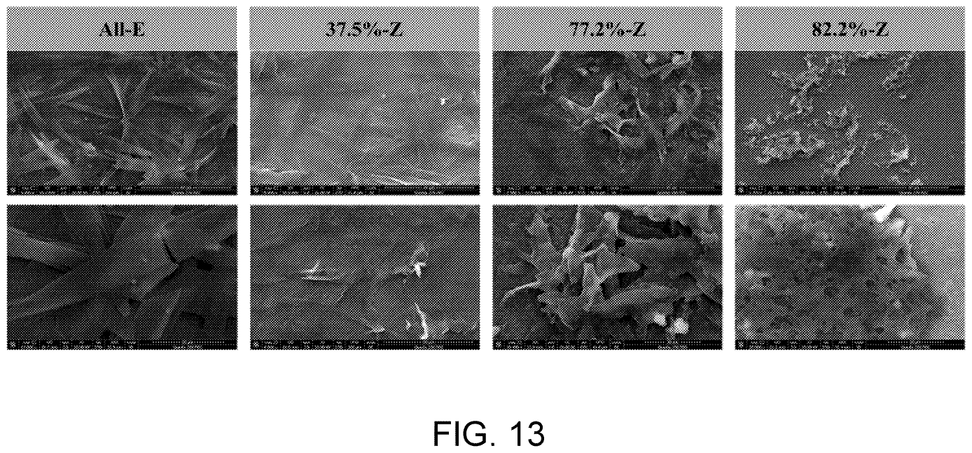

As shown in FIGS. 11 and 12, when the catalytic time was 0.5 h, the isomerization rate was 22.9% and the retention rate was 96.9%; when the catalytic time was 1 h, the isomerization rate was 82.2% and the retention rate was 90.3%; and when the catalytic time was extended to 2 h, the cis-isomerization rate of lycopene was 63.1%, while the retention rate was decreased to 22.2%. Therefore, when the catalytic time was 1 h, the isomerization effect was optimal.

SEM analysis of samples with different cis-lycopene contents:
(1) The sample solutions of standard trans-lycopene, lycopene product obtained in Comparative Example 4 (37.5-Z), lycopene product obtained in Comparative Example 5 (77.2%-Z), and lycopene product obtained in Example 2 (82.2%-Z) in the above experiments were selected and dropped onto silicon wafers and dried at 15° C.; and
(2) the microscopic morphology of lycopene with different cis-lycopene contents was further observed using scanning electron microscopy (SEM).

As shown in FIG. 13, trans-lycopene formed sheet-like crystals, while as the proportion of cis-lycopene in the catalytic system was gradually increased, lycopene became less prone to crystallization and was gradually transformed into an amorphous form.

XRD analysis of samples with different cis-lycopene contents:
(1) The sample solutions of standard trans-lycopene, lycopene product obtained in Comparative Example 3 (37.5%-Z), lycopene product obtained in Comparative Example 7 (77.2%-Z), and lycopene product obtained in Example 1 (82.2%-Z) in the above experiments were selected and subjected to rotary evaporation at 4° C. to obtain powders; and
(2) the crystal structures of lycopene with different cis-lycopene contents were further analyzed by X-ray diffraction (XRD).

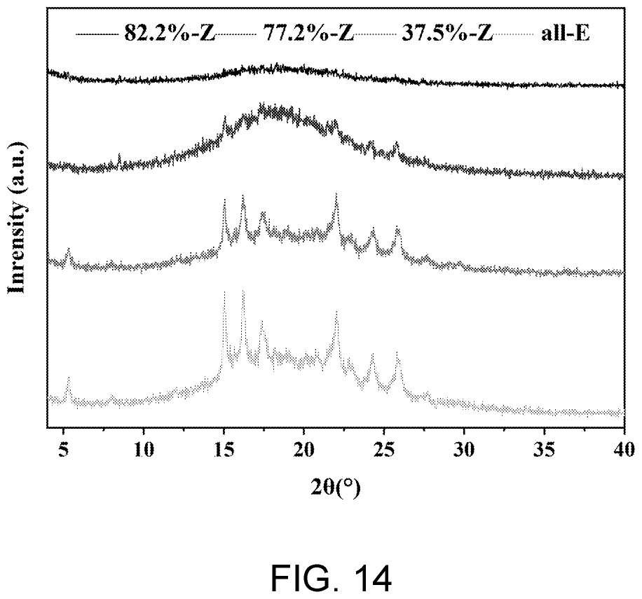

As shown in FIG. 14, trans-lycopene exhibits a crystalline form; however, as the proportion of cis-lycopene in the catalytic system was gradually increased, the crystallinity of lycopene was gradually decreased. This is attributed to the fact that when the content of Z-isomers increases, significant steric hindrance is generated, thereby reducing the potential π-π attractive interactions, which in turn leads to a decrease in crystallinity.

Therefore, the highly efficient and stable method for isomerization of lycopene of the present disclosure improves the isomerization efficiency and retention rate of trans-lycopene by regulating the occurrence form of water molecules in the catalytic medium, selecting specific catalysts, and adjusting the catalytic concentration and catalytic time of the catalyst, thereby exhibiting great application prospects.

What is claimed is:

1. A highly efficient and stable method for cis-isomerization of lycopene, wherein a bimetallic metal-organic framework material is used as a catalyst to catalyze lycopene to undergo cis-isomerization.

2. The method according to claim 1, wherein the bimetallic metal-organic framework material is formed by doping a zirconium-based organic framework material with a metal material, a crystal structure of the bimetallic metal-organic framework material is octahedral and formed by coordination between an organic ligand tetrafluoroterephthalic acid and metal ions, and the metal ions are zirconium ions, copper ions, zinc ions, or iron ions; among the metal ions, copper ions, zinc ions, or iron ions are doped metal ions, and a molar amount of the doped metal ions is 20%-50% of a total molar amount of the metal ions.

3. The method according to claim 1, wherein the zirconium-based organic framework material comprises UIO-66 or MOF-801, and the bimetallic metal-organic framework material is Zn_{1/5}—UIO-66, Zn_{1/4}—UIO-66, Zn_{1/2}—UIO-66, Cu_{1/5}—UIO-66, Cu_{1/4}—UIO-66, Cu_{1/2}—UIO-66, Fe_{1/5}—UIO-66, Fe_{1/4}—UIO-66, Fe_{1/2}—UIO-66, or Cu-MOF-801.

4. The method according to claim 1, wherein a method for preparing the bimetallic metal-organic framework material comprises the following steps:
(1) dissolving zirconyl nitrate together with one of copper nitrate trihydrate, zinc nitrate hexahydrate, and iron nitrate nonahydrate in a solution of methanol and formic acid, and stirring for dissolution to obtain a mixed solution, wherein a molar ratio of the zirconyl nitrate to one of the copper nitrate trihydrate, the zinc nitrate hexahydrate, and the iron nitrate nonahydrate is 4:1 to 1:1, and a volume ratio of the methanol to the formic acid is 5:1 to 2:1;
(2) adding tetrafluoroterephthalic acid into the mixed solution, and stirring at a temperature of 30° C.-50° C. for 10 h-14 h; and
(3) subjecting a solid material obtained to centrifugal washing and vacuum drying to obtain the bimetallic metal-organic framework material, wherein methanol is used as a washing agent, and the centrifugation is performed at a rotating speed of 3000 rpm/min for 10 min at a temperature of 10° C.-20° C.; the vacuum drying is performed at a temperature of 45° C.-60° C. for 8 h-10 h under a drying pressure of −0.09 MPa to −0.1 MPa.

5. The method according to claim 1, wherein the method specifically comprises using the bimetallic metal-organic framework material and a lycopene solution, stirring, performing heating under reflux in a water bath, and allowing to react in the dark; and filtering a lycopene mixed solution after the reaction to obtain a product, wherein a ratio of an amount of the bimetallic metal-organic framework material used to lycopene used is 4:1 to 2:1, the heating is performed at a temperature of 25° C.-50° C. for 0.5 h-2 h, magnetic stirring is employed, heating under reflux in a water bath is performed, and the reaction is performed in the dark.

6. The method according to claim 1, wherein a catalytic medium is further added to form a catalytic system to perform cis-isomerization preparation of lycopene.

7. The method according to claim 6, wherein the catalytic medium is confined water, and a volume percentage of the catalytic medium in the catalytic system is 1%-2%.

8. The method according to claim 6, specifically comprising:
(1) dissolving trans-lycopene in acetone, adding the bimetallic metal-organic framework material into a reaction system, and adding the catalytic medium into the reaction system to form a catalytic system;
(2) subjecting the above catalytic system to water-bath heating and stirring; and
(3) centrifuging a solution after completion of catalysis to recover a catalyst precipitate, thereby obtaining a solution of cis-lycopene in acetone and completing isomerization of lycopene.

9. The method according to claim 8, wherein the bimetallic metal-organic framework material is a Cu-doped MOF-801 transition metal-based nanomaterial.

10. The method according to claim 8, wherein the water-bath heating and stirring are performed at a temperature of 30° C.-40° C.
